(19) United States
(12) Patent Application Publication
(10) Pub. No.: US 2024/0008948 A1
(43) Pub. Date: Jan. 11, 2024

(54) SYSTEM FOR PERFORMING REAL-TIME AORTIC VALVE DIAMETER MEASUREMENT

(71) Applicant: MiRus LLC, Marietta, GA (US)

(72) Inventor: Daniel Burnham, Marietta, GA (US)

(21) Appl. No.: 18/219,798

(22) Filed: Jul. 10, 2023

Related U.S. Application Data
(60) Provisional application No. 63/359,401, filed on Jul. 8, 2022.
(60) Provisional application No. 63/411,266, filed on Sep. 29, 2022.

(51) Int. Cl. A61B 90/00; A61M 25/10

(52) U.S. Cl. CPC A61B 90/06; A61M 25/10; A61M 25/10187; A61M 2205/3368; A61M 2205/3331; A61B 2090/061

(57) ABSTRACT

A method and apparatus for measuring the inflation diameter, cross-sectional area, and/or volume of an inflatable balloon on a balloon catheter.

The present application claims priority on U.S. Provisional Application Ser. No. 63/359,401 filed Jul. 8, 2022 and 63/411,266 filed Sep. 29, 2022, both of which are incorporated herein by reference.

### FIELD OF THE DISCLOSURE

The disclosure relates generally to medical devices and medical device applications, more particularly to a medical device used in the treatment of structural heart disease and cardiovascular implants, and even more particularly to a device used to facilitate in the implantation of a prosthetic heart valve or a transcatheter heart valve or balloon valvuloplasty.

### BACKGROUND OF THE DISCLOSURE

Many cardiovascular procedures such as the implantation of stents and heart valves and interventional cardiac procedures such as valvuloplasty require insertion of a balloon expandable catheter and expansion of balloon at the treatment site. These consist of deflated balloon devices which may be combined with a crimped device such as stents, expandable heart valves, and the like are inserted into a patient via the vascular system of a patient and then expanded at the treatment site. These devices are typically crimped onto the catheter prior to insertion into a patient.

Medical devices such as transcatheter aortic valves (TAVs) represent a significant advancement in prosthetic heart valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; and US Publication No. 2020/0405482, all of which are incorporated fully herein by reference.

A TAV is designed to be compressed into a small diameter catheter and remotely placed within a patient's diseased aortic valve to take over the function of the native valve. Some TAVs are balloon-expandable, while others are self-expandable. In both cases, the TAVs are deployed within a calcified native valve that is forced permanently open and becomes the surface against which the stent is held in place by friction. TAVs can also be used to replace failing bioprosthetic or transcatheter valves, commonly known as a valve in valve procedure. Major TAVR advantages to the traditional surgical approaches include refraining using cardiopulmonary bypass, aortic cross-clamping, and sternotomy that significantly reduces patients' morbidity.

However, several complications are associated with current TAV devices such as mispositioning, crimp-induced leaflet damage, paravalvular leak, thrombosis, conduction abnormalities, and prosthesis-patient mismatch. These complications are potentially associated with the calcification landscape of the native valve, geometric and mechanical properties of the aortic root, blood biochemistry and coagulability associated with the patient, and concomitant conditions such as hypertension, coronary artery disease, heart failure, etc.

During a transaortic valve replacement, the valve sizing and expansion is key to providing a good clinical outcome for the patient. During the procedure, a clinician implants a balloon-expandable stent frame by use of a catheter inserted in the femoral artery. At the location of the aorta, the clinician expands the stent to match the diameter of the vessel walls. Insufficient sizing can lead to serious adverse events in clinical outcomes, including perivalvular leakages (PVL). Overexpansion of the valve may lead to damage to the cardiac muscle about the heart valve and could in some cases lead to the requirement of a cardiac pacemaker for pacing of the heart.

A valvuloplasty is a procedure to repair a heart valve that has a narrowed opening. In a narrowed heart valve (stenosis), the valve flaps (leaflets) may become thick or stiff and fuse together, thus reducing blood flow through the valve. A valvuloplasty may improve blood flow through the heart valve. During the procedure, a catheter tipped with a balloon is inserted into a body passageway and guided to the narrowed valve in the heart. Once the balloon is in position, the balloon is inflated to widen the valve, thereby improving blood flow through the heart valve. Thereafter, the balloon is deflated and the catheter with balloon is removed from the heart valve. One issue associated with valvuloplasty is that overexpansion of the balloon may lead to damage to the cardiac muscle about the heart valve and could, in some cases lead, to the requirement of a cardiac pacemaker for pacing of the heart.

In view of the current state of the art of prosthetic heart valves or transcatheter heart valves, there is a need for a medical device that can provide real-time measurement of the diameter of the prosthetic heart valve or a transcatheter heart valve as such device is expanded in a heart valve to facilitate in the proper expansion of the medical device.

### SUMMARY OF THE DISCLOSURE

The disclosure relates generally to medical devices and medical device applications, more particularly to a medical device used in the treatment of structural heart disease and cardiovascular implants, particularly to a device used to facilitate in the implantation of an expandable device in the cardiovascular system, and more particularly to a device that is used to facilitate in the implantation of a prosthetic heart valve or a transcatheter heart valve in a heart to facilitate in a balloon valvuloplasty. In one non-limiting embodiment, there is provided a device that can be used in a stent or a prosthetic heart valve or a transcatheter heart valve procedure to measure the diameter of the expanded stent, the expanded prosthetic heart valve, or a transcatheter heart valve as the stent or the prosthetic heart valve or a transcatheter heart valve is expanded in a blood vessel, heart valve, etc. Such a device, when used with a prosthetic heart valve or a transcatheter heart valve, can a) minimize perivalvular leakages (PVL) about the prosthetic heart valve or a transcatheter heart valve, b) prevent overexpansion of the prosthetic heart valve or a transcatheter heart valve in the heart, and/or c) minimize or prevent damage to the region of the heart in which the prosthetic heart valve or a transcatheter heart valve has been expanded. Such a device, when used with a stent, can a) ensure proper expansion of the stent in a blood vessel, b) prevent overexpansion of the stent in a blood vessel, and/or c) minimize or prevent damage to the blood vessel from the expanded stent.

In one non-limiting aspect of the present disclosure, there is provided a diameter measurement device (DMD) that can be used to measure in real-time a) the diameter of a medical device that is expanded in the cardiovascular system, b) the cross-sectional area of a medical device that is expanded in the cardiovascular system, and/or c) the volume of a medical device that is expanded in the cardiovascular system.

In another and/or alternative non-limiting aspect of the present disclosure, there is provided a DMD that can be used to measure in real-time the diameter and/or cross-sectional area of a stent that is expanded in a blood vessel.

In another and/or alternative non-limiting aspect of the present disclosure, there is provided a DMD that can be used to measure in real-time the diameter and/or cross-sectional area of a prosthetic heart valve or a transcatheter heart valve as the prosthetic heart valve or a transcatheter heart valve is that is expanded in a heart valve.

In another and/or alternative non-limiting aspect of the present disclosure, there is provided a DMD that can be used to measure in real-time the diameter and/or cross-sectional area of an inflation device (e.g., inflatable balloon, etc.) that is expanded and/or contracted in a blood vessel.

In another and/or alternative non-limiting aspect of the present disclosure, there is provided a DMD that uses impedance planimetry to determine the diameter and/or cross-sectional area of the medical device (e.g., stent, prosthetic heart valve or a transcatheter heart valve, inflatable balloon, etc.) as the medical is expanded in a blood vessel, heart valve, etc., by measuring the volume of the inflatable balloon used to expand the medical device. The impedance planimetry can include one or more conducting electrodes located in the interior of the inflatable balloon. In one non-limiting configuration, impedance planimetry can be used in an array form to provide diameter measurements through an inflatable balloon for use in determining the diameter and/or cross-sectional area of the medical device in real time as the medical device is expanded.

In another and/or alternative non-limiting aspect of the present disclosure, there is provided a DMD that uses impedance planimetry to determine the diameter and/or cross-sectional area of the medical device and at least a portion of the DMD is located in the interior of an inflatable balloon of a balloon catheter. In such a non-limiting arrangement, multiple excitation electrodes are placed along the catheter shaft. A plurality of the excitation electrodes are located in the interior of the expandable balloon. One or more of the excitation electrode can be located remotely from the inflatable balloon. One or more electrically-conducing wires are used to energize the excitation electrodes. The one or more electrically-conducting wires extend from the inflatable balloon located at one end of the catheter to a location that is remote from the inflatable balloon (e.g., front portion of the catheter, etc.). The excitation electrodes can be fabricated by various means (e.g., cut hypo tube, foil wrapping, or other means known to the art).

In another and/or alternative non-limiting aspect of the present disclosure, there is provided a DMD that uses impedance planimetry to determine the diameter and/or cross-sectional area of the medical device and wherein at least two excitation electrodes are located in the interior of the inflatable balloon and at least two sensor electrodes are also located in the interior of the inflatable balloon. In one non-limiting arrangement, first and second excitation electrodes are placed in the interior of the inflatable balloon and first and second sensor electrodes are positioned between the first and second excitation sensors. In another non-limiting arrangement, the first and second sensor electrodes are spaced a closer distance to one another than a spacing distance of either of the sensor electrodes to either of the excitation electrodes. As can be appreciated, more than two excitation electrodes can be located in the interior of the inflatable balloon, and/or more than two sensor electrodes can be located in the interior of the inflatable balloon. The size of the circuit that includes the excitation and sensor electrodes in the inflatable balloon is non-limiting. Generally, the length of the circuit is 0.05-6 inches (and all values and ranges therebetween). Generally, the length of the circuit that located in the inflatable balloon is less than or equal to the longitudinal length of the inflatable balloon; however, this is not required.

In another and/or alternative non-limiting aspect of the present disclosure, there is provided a DMD that uses impedance planimetry to determine the diameter and/or cross-sectional area of the medical device and wherein the excitation electrodes and/or the sensor electrodes can optionally be ring-shaped or semi-circular-shaped electrodes. As can be appreciated, other shaped electrodes can be used.

In another and/or alternative non-limiting aspect of the present disclosure, there is provided a DMD that uses impedance planimetry to determine the diameter and/or cross-sectional area of the medical device and wherein the plurality of excitation electrodes are configured to produce an AC signal which can be, but is not limited to, a sinusoidal wave, a square wave, or a triangle wave. The spacing of the excitation electrodes from one another can be a function of the expanded diameter and/or cross-sectional area of the balloon to provide a uniform current density within the inflatable balloon. The sensor electrodes can be selected differentially through a multiplexed input to provide differential electrode potentials. This multiplexing mechanism can be used to measure each segment individually and increase the resolution of the system by averaging adjacent segments to provide a sub segment measurement. The sensed differential electrode potentials can be used to determine the expanded diameter and/or cross-sectional area of the inflatable balloon, which in turn is used to determine the expanded diameter and/or cross-sectional area of the medical device. In one non-limiting arrangement, a plurality of sensor electrodes and excitation sensors are separated by a fixed distance and are connected via wires to a voltage source. A constant AC current source is supplied to the wires and an electric field is generated in a conductive medium that is contained in the inflatable balloon. The diameter of the inflatable balloon can be calculated by the following formula:

V/I=R=L/Aσ=L/(π(D/2)²×σ)

wherein R is a resistance (impedance). R is equal to V/I. V is the AC voltage and I is the AC current. I is a known AC current and V can be measured across the excitation electrodes by use of the sensor electrodes. L is a fixed distance between the excitation electrodes. σ is a medium (e.g., saline solution, saline and dye mixture, etc.) having a conductivity at a certain temperature. D is the diameter of the inflatable balloon. A is the cross-sectional area of the inflatable balloon which assumes that the cross-sectional area is uniform along the longitudinal length of the inflatable balloon as the inflatable balloon is inflated. This equation relies on the relationship of a conductive body of fluid and the resistance being inversely proportional to the conductivity which is influenced by cross-sectional area of the inflatable balloon and an ion concentration (conductance) of the fluid medium.

In another and/or alternative non-limiting aspect of the present disclosure, there is provided a DMD that uses impedance planimetry to determine the diameter and/or cross-sectional area of the medical device and wherein the inflatable balloon can be optionally divided into segments and measured by the differential pairs of the excitation and sensor electrodes to allow for a measurement of the envelope of the diameter of the inflatable balloon.

In another and/or alternative non-limiting aspect of the present disclosure, there is provided a DMD that uses impedance planimetry to determine the diameter and/or cross-sectional area of the medical device and wherein the circuit uses a constant voltage source across a known calibration resistor with high precision by injecting a voltage waveform. The impedance measurements are then sequenced across electrodes for segmental impedance measurements using the calibrated current value and voltages determined from each segment. The voltage waveform may be an AC signal such as, but not limited to, a sine wave, a triangle waveform, or a square waveform. The voltage waveform may also or alternatively be DC that is measured periodically.

In another and/or alternative non-limiting aspect of the present disclosure, there is provided a DMD that uses impedance planimetry to determine the diameter and/or cross-sectional area of the medical device and wherein the circuit used for the impedance planimetry includes the use of an excitation source producing an AC signal of 50 hz to 50 khz (and all values and ranges therebetween). The AC signal of the sensor electrodes can be conditioned to extract a peak value. The extraction of a peak value can be accomplished by optionally using an RMS circuit detector, or a peak detector for the AC waveform. The conditioning circuit can be first buffered then optionally multiplexed to combine differential pairs or could be sampled individually. The use of multiplexing can be used to reduce the size of the circuit. The sensed signal can be rectified using a diode and the current peak is held using a capacitor, thereby producing a DC signal. The DC signal can then be optionally fed into an analog-to-digital converter. The DC signal can optionally be subjected to additional filtering to reduce or eliminate unwanted noise. These signal filtering techniques may use DSP or analog signal conditioners and rely on techniques known in the art. The digitized signal can optionally be sent to a microcontroller to extract the average peak of the signal and compute the resultant diameter and/or cross-sectional area of the inflatable balloon.

In another and/or alternative non-limiting aspect of the present disclosure, there is provided a DMD that uses impedance planimetry to determine the diameter and/or cross-sectional area of the medical device and includes circuitry to transmit data (e.g., inflatable balloon diameter information, etc.) by wire or wirelessly (e.g., Bluetooth®, RF, etc.) to a remote location (e.g., a control unit, monitor, computer, network, etc.). The calculated diameter and/or cross-sectional area values of the inflatable balloon can be displayed in real-time on a monitor during the inflation of the inflatable balloon, and/or can be stored.

In another and/or alternative non-limiting aspect of the present disclosure, there is provided a DMD that uses impedance planimetry to determine the diameter and/or cross-sectional area of the medical device and wherein a temperature sensor is optionally positioned at or near the inflatable balloon. When the inflatable balloon is inserted into the vascular of a patient, the temperature of the inflatable balloon will change. Also, when a medium such as a saline solution is inserted into the inflatable balloon to cause inflation of the inflatable balloon, the temperature of the saline solution will change between the time the saline solution is first introduced into the catheter at one end and the time that saline solution flow through the catheter and inflates the inflatable balloon. The optional temperature sensor is used to compensate for a change in a (medium conductivity) for a certain measured temperature of the medium in the inflatable balloon. The location of the temperature sensor on the catheter is non-limiting (e.g., inside the inflatable balloon, at the end of the balloon catheter, at the beginning of the balloon catheter, etc.).

In another and/or alternative non-limiting aspect of the present disclosure, there is provided a DMD that uses impedance planimetry to determine the diameter and/or cross-sectional area of the medical device and wherein a flow sensor is used to provide information about total volume of fluid into the inflatable balloon. This sensor can be used to monitor the fluid flow rate and/or total fluid volume delivered to the inflatable balloon to provide additional information for determining the diameter and/or cross-sectional area of the inflatable balloon based on known relationships between the diameter and/or cross-sectional area of the inflatable balloon and the volume of the inflatable balloon.

In another and/or alternative non-limiting aspect of the present disclosure, there is provided a DMD that uses impedance planimetry to determine the diameter and/or cross-sectional area of the medical device and wherein a pressure sensor is located either distally in the balloon or proximally in the inflating system and is used to assist in determining the diameter of the balloon by known pressure volume compliance calculations for the balloon.

In another and/or alternative non-limiting aspect of the present disclosure, there is provided a DMD that uses impedance planimetry to determine the diameter and/or cross-sectional area of the medical device and wherein a reference container is used to provide reference information that can be used to further adjust the calculated diameter of the inflatable balloon. Because the fluid concentration of the medium may not be known at the time of a procedure, a reference measurement can be taken at a known diameter to calculate the a (medium conductivity) for a certain medium at a certain temperature. Prior to the inflation of the inflatable balloon in the body passageway (e.g., blood vessel, etc.) or heart, the fluid medium that is to be used for the medical procedure can be included into a reference tube (e.g., a cylindrical tube, etc.) that has a fixed (e.g., constant diameter along the longitudinal length of the tube) and known diameter. The fixed diameter of the reference tube can be a fixed diameter that is close to the anticipated diameter of the inflatable balloon when fully inflated in the body passageway or heart; however, this is not required. The reference tube can optionally include a temperature monitor to measure the temperature of the fluid medium in the reference tube. The electrode configuration on the reference tube is the same or substantially the same as the electrode configuration in the inflatable balloon when the inflatable balloon is inflated. After the fluid medium fills the reference tube, the a (medium conductivity) for the medium can be calculated based on the known AC current and diameter and/or cross-sectional area of the reference tube. The measured temperature in the reference tube can optionally be used to determine a temperature adjustment of the a (medium conductivity) based on the temperature of the medium. The reference tube can optionally be configured to be in fluid communication with the inflatable balloon during the medical procedure of inserting the medical device in the patient. In such non-limiting configuration, the composition of the medium in the inflatable balloon and the reference tube is the same or substantially the same, thus real-time compensation of the a (medium conductivity) can be obtained during the medical procedure, which in turn results in a more accurate diameter calculation of the inflatable balloon during the inflation of the inflatable balloon. Also, when the balloon catheter includes a temperature sensor at or near the inflatable balloon, the measure temperature can be used to further provide real-time compensation of the a (medium conductivity) during the medical procedure. In one non-limiting embodiment, the temperature of the fluid medium in the reference tube can optionally be adjusted to be the same or similar to the temperature provided by the temperature sensor at or near the inflatable balloon to provide a more accurate real-time compensation of the a (medium conductivity) during the medical procedure, which in turn results in a more accurate diameter and/or cross-sectional area calculation of the inflatable balloon during the inflation of the inflatable balloon. The calculation of the a (medium conductivity) can a) optionally be calculated for multiple concentrations of saline or with a contrast medium that is used to improve visibility in the x-ray prior to the medical procedure, b) optionally be calculated once per fluid load during the medical procedure, and/or c) optionally be calculated in real time. The calculation of a (medium conductivity) and/or calculated diameter and/or cross-sectional area of the inflatable balloon can be accomplished by use of a microcontroller, and such information can optionally be transmitted by wire or wirelessly to a monitor and/or storage device (e.g., smart phone, computer, network, tablet, etc.) real-time or near real-time during the medical procedure.

In another and/or alternative non-limiting aspect of the present disclosure, there is the provision of a diameter measurement device system (DMDS) configured to measure a) a diameter of at least a portion or all of a medical device that is expanded in a body passageway, b) a cross-sectional area of at least a portion or all of the medical device that is expanded in the body passageway, and/or c) a volume of at least a portion or all of the medical device that is expanded in the body passageway.

In another and/or alternative non-limiting aspect of the present disclosure, there is the provision of a diameter measurement device system (DMDS) for use with a medical device, and wherein the medical device optionally includes a balloon catheter, and wherein the balloon catheter optionally includes a catheter body having a distal portion, a central portion and proximal portion; and wherein the balloon catheter optionally includes the inflatable balloon connected at or near said distal portion.

In another and/or alternative non-limiting aspect of the present disclosure, there is the provision of a diameter measurement device system (DMDS), and wherein the DMDS includes a plurality of excitation electrodes and a plurality of sensor electrodes; and wherein the plurality of excitation electrodes and the plurality of sensor electrodes are positioned inside the inflatable balloon; and wherein the plurality of excitation electrodes and the plurality of sensor electrodes are spaced from one another.

In another and/or alternative non-limiting aspect of the present disclosure, there is the provision of a diameter measurement device system (DMDS), and wherein the DMDS includes a plurality of wires that provide current to a plurality of excitation electrodes and a plurality of sensor electrodes; and wherein each of the plurality of excitation electrodes and the plurality of sensor electrodes are partially or fully encircle the plurality of wires.

In another and/or alternative non-limiting aspect of the present disclosure, there is the provision of a diameter measurement device system (DMDS), and wherein the DMDS is configured to measure a change in the impedance-derived cross-sectional area and pressure of the inflatable balloon as the inflatable balloon is inflated to determine a) the diameter of at least a portion of the medical device that is expanded in the body passageway, b) the cross-sectional area of at least said portion of the medical device that is expanded in the body passageway, and/or c) the volume of at least said portion of the medical device that is expanded in the body passageway.

In another and/or alternative non-limiting aspect of the present disclosure, there is the provision of a diameter measurement device system (DMDS), and wherein the medical device includes a stent or prosthetic heart valve that is at least partially positioned about the inflatable balloon.

In another and/or alternative non-limiting aspect of the present disclosure, there is the provision of a diameter measurement device system (DMDS), and wherein the distal portion of the catheter includes a temperature sensor.

In another and/or alternative non-limiting aspect of the present disclosure, there is the provision of a diameter measurement device system (DMDS), and wherein the DMDS includes multiplexed inputs.

In another and/or alternative non-limiting aspect of the present disclosure, there is the provision of a diameter measurement device system (DMDS), and wherein the DMDS includes a wireless transmitter that wirelessly transmits data to a remote location so that data can be stored and/or displayed at a remote location.

In another and/or alternative non-limiting aspect of the present disclosure, there is the provision of a diameter measurement device system (DMDS), and wherein the DMD includes a reference container; said reference container has a) fixed and constant cross-sectional area along a longitudinal length of said reference container, b) a fixed and constant volume, c) a fixed and constant cross-sectional area along a longitudinal length of said reference container, and/or d) a fixed and constant cross-sectional shape along a longitudinal length of said reference container; said reference container includes a plurality of reference electrodes located in said reference container; a number, orientation, and/or spacing of said plurality of reference electrodes in said reference container is the same or substantially the same as a number, orientation, and/or spacing of said excitation and senor electrodes in said inflatable balloon.

In another and/or alternative non-limiting aspect of the present disclosure, there is the provision of a diameter measurement device system (DMDS), and wherein the reference tube has the same or similar longitudinal length as the inflatable balloon.

In another and/or alternative non-limiting aspect of the present disclosure, there is the provision of a diameter measurement device system (DMDS), and wherein the reference tube is used to calculate a medium conductivity of a medium; and wherein the medium is optionally used to inflate the inflatable balloon.

In another and/or alternative non-limiting aspect of the present disclosure, there is the provision of a diameter measurement device system (DMDS), and wherein the medium includes a saline solution.

In another and/or alternative non-limiting aspect of the present disclosure, there is the provision of a diameter measurement device system (DMDS), and wherein a constant voltage source having a high precision waveform is used across a known calibration resistor and impedance measurements are optionally sequenced across electrodes for segmental impedance measurements using the calibrated current value and voltages determined from each segment.

In another and/or alternative non-limiting aspect of the present disclosure, there is the provision of a diameter measurement device system (DMDS), and further including a flow sensor to provide information about total volume of fluid in the inflatable balloon, and wherein the flow sensor can be optionally used to monitor a fluid flow rate and/or a total fluid volume delivered to the inflatable balloon and provide additional information for determining the diameter of at least a portion of the medical device, the cross-sectional area of at least a portion of the medical device, and/or the volume of at least a portion of the medical device based on known relationships of the inflatable balloon.

In another and/or alternative non-limiting aspect of the present disclosure, there is the provision of a diameter measurement device system (DMDS), and further including a pressure sensor located distally in the inflation balloon and/or proximally in an inflating system, and wherein the pressure sensor is optionally used to facilitate determining the diameter, the cross-sectional area, and/or the volume of at least a portion of the medical device by known pressure volume compliance calculations for the inflatable balloon and/or the inflating system.

In another and/or alternative non-limiting aspect of the present disclosure, there is the provision of a diameter measurement device system (DMDS), and of a method for measuring a) a diameter of at least a portion of a medical device that is expanded in a body passageway, b) a cross-sectional area of at least a portion of the medical device that is expanded in the body passageway, and/or c) a volume of at least a portion of the medical device that is expanded in the body passageway, wherein the method comprises A) providing the medical device; and wherein the medical device includes a balloon catheter; and wherein the balloon catheter includes a catheter body having a distal portion, a central portion, and proximal portion; and wherein the balloon catheter includes an inflatable balloon connected at or near said distal portion; B) providing a diameter measurement device (DMD) configured to measure i) the diameter of at least a portion of the medical device that is expanded in the body passageway, ii) a cross-sectional area of at least a portion of the medical device that is expanded in the body passageway, and/or iii) a volume of at least a portion of the medical device that is expanded in the body passageway; and wherein the DMD includes a plurality of excitation electrodes and a plurality of sensor electrodes; and wherein the plurality of excitation electrodes and the plurality of sensor electrodes are positioned inside the inflatable balloon; and wherein the plurality of excitation electrodes and the plurality of sensor electrodes spaced from one another; C) inserting the distal portion and a portion of the central portion of the catheter into a body passageway; D) moving the distal portion of the catheter in the body passageway until the distal portion is positioned at a treatment area; E) expanding the inflatable balloon at the treatment area; and F) measuring a change in the impedance derived cross-sectional area and pressure of the inflatable balloon by the DMD to determine at the treatment area I) the diameter of at least a portion or all of the medical device that is expanded in a body passageway, II) the cross-sectional area of at least a portion or all of the medical device that is expanded in the body passageway, and/or III) the volume of at least a portion or all of the medical device that is expanded in the body passageway.

In another and/or alternative non-limiting aspect of the present disclosure, there is the provision of a diameter measurement device system (DMDS), and of a method for measuring a) a diameter of at least a portion or all of a medical device that is expanded in a body passageway, b) a cross-sectional area of at least a portion or all of the medical device that is expanded in the body passageway, and/or c) a volume of at least a portion or all of the medical device that is expanded in the body passageway, wherein the measuring a) the diameter of at least a portion or all of the medical device that is expanded in the body passageway, b) the cross-sectional area of at least a portion or all of the medical device that is expanded in the body passageway, and/or c) the volume of at least a portion or all of the medical device that is expanded in the body passageway is in real time or near real time.

In another and/or alternative non-limiting aspect of the present disclosure, there is the provision of a diameter measurement device system (DMDS), and of a method for measuring a) a diameter of at least a portion or all of a medical device that is expanded in a body passageway, b) a cross-sectional area of at least a portion or all of the medical device that is expanded in the body passageway, and/or c) a volume of at least a portion or all of the medical device that is expanded in the body passageway, wherein the step of measuring a change in the impedance includes impedance planimetry, wherein a constant voltage source having a high precision waveform is used across a known calibration resistor, and impedance measurements are optionally sequenced across electrodes for segmental impedance measurements using the calibrated current value and voltages determined from each segment.

These and other aspects and advantages will become apparent from the discussion of the distinction between the disclosure and the prior art and when considering the non-limiting embodiment illustrated in the accompanying drawing.

### BRIEF DESCRIPTION OF THE DRAWING

The above and other features and advantages of the disclosure will become apparent from a consideration of the subsequent detailed description presented in connection with the accompanying drawings in which:

FIG. 1 illustrates a block diagram of a catheter that includes a diameter measurement device (DMD) that can be used to measure in real-time a) the diameter of a medical device that is expanded in the cardiovascular system, b) the cross-sectional area of a medical device that is expanded in the cardiovascular system, and/or c) the volume of a medical device that is expanded in the cardiovascular system.

### DETAILED DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

These and other advantages will become apparent to those skilled in the art upon the reading and following of this description.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Some portions of the detailed description herein are presented in terms of algorithms and symbolic representations of operations on data bits performed by conventional computer components, including a central processing unit (CPU), memory storage devices for the CPU, and connected display devices. These algorithmic descriptions and representations are the means used by those skilled in the data processing arts to most effectively convey the substance of their work to others skilled in the art. An algorithm is generally perceived as a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be understood, however, that all of these and similar terms are to be associated with the appropriate physical quantities and are merely convenient labels applied to these quantities. Unless specifically stated otherwise, as apparent from the discussion herein, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The exemplary embodiment also relates to an apparatus for performing the operations discussed herein. This apparatus may be specially constructed for the required purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus.

The algorithms and displays presented herein are not inherently related to any particular computer or other apparatus. Various general-purpose systems may be used with programs in accordance with the teachings herein, or it may prove convenient to construct more specialized apparatus to perform the methods described herein. The structure for a variety of these systems is apparent from the description above. In addition, the exemplary embodiment is not described with reference to any particular programming language. It will be appreciated that a variety of programming languages may be used to implement the teachings of the exemplary embodiment as described herein.

A machine-readable medium includes any mechanism for storing or transmitting information in a form readable by a machine (e.g., a computer). For instance, a machine-readable medium includes read only memory (“ROM”), random access memory (“RAM”), magnetic disk storage media, optical storage media, flash memory devices, and electrical, optical, acoustical or other form of propagated signals (e.g., carrier waves, infrared signals, digital signals, etc.).

The methods illustrated throughout the specification, may be implemented in a computer program product that may be executed on a computer. The computer program product may comprise a non-transitory computer-readable recording medium on which a control program is recorded, such as a disk, hard drive, or the like. Common forms of non-transitory computer-readable media include, for example, floppy disks, flexible disks, hard disks, magnetic tape, or any other magnetic storage medium, CD-ROM, DVD, or any other optical medium, a RAM, a PROM, an EPROM, a FLASH-EPROM, or other memory chip or cartridge, or any other tangible medium from which a computer can read and use.

One non-limiting exemplary embodiment is described herein. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

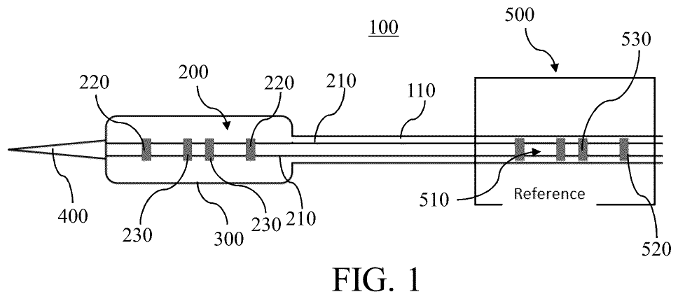

Referring now to FIG. 1, there is illustrated a block diagram of a catheter 100 that includes a diameter measurement device (DMD) 200 that can be used to measure in real-time a) the diameter of a medical device that is expanded in the cardiovascular system, b) the cross-sectional area of a medical device that is expanded in the cardiovascular system, and/or c) the volume of a medical device that is expanded in the cardiovascular system. Positioned on the distal portion of catheter 100 is an inflatable balloon 300.

DMD 200 includes a plurality of wires 210 that conducts current from a power source (not shown). Positioned inside inflatable balloon 300 are a plurality of excitation electrodes 220 and a plurality of sensor electrodes 230. The two sensor electrodes 230 are illustrated as being positioned between excitation electrodes 220; however, this is not required.

A temperature sensor 400 is optionally positioned at or near the distal tip of catheter 100.

Positioned at or near the posterior end of catheter 100 is an optional reference tube 500.

Reference tube 500 can be optionally fluidly connected to the body 110 of the catheter 100. Reference tube 500 is a fixed and constant diameter tube and/or a fixed and constant cross-sectional shape and size along a longitudinal length of the tube (e.g., cylindrical tube, etc.) that includes a circuit 510 that is the same or similar to DMD 200 inside inflatable balloon 300. As such circuit 510 includes two excitation electrodes 520 and two sensor electrodes 530 positioned between the two excitation electrodes.

Body 110 can includes one or more internal passageways that extend along the longitudinal length of body 110. The one or more internal passageways can include wires 210. The one or more internal passageways include a fluid passageway that allows fluid (e.g., saline, etc.) to flow from the proximal end of the catheter to the distal portion of the catheter to enable the inflatable balloon to be inflated and deflated.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall there between. The invention has been described with reference to the preferred embodiments. These and other modifications of the preferred embodiments as well as other embodiments of the invention will be obvious from the disclosure herein, whereby the foregoing descriptive matter is to be interpreted merely as illustrative of the invention and not as a limitation. It is intended to include all such modifications and alterations insofar as they come within the scope of the appended claims.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A method for measuring a) a diameter of at least a portion or all of a medical device that is expanded in a body passageway, b) a cross-sectional area of at least a portion or all of said medical device that is expanded in said body passageway, and/or c) a volume of at least a portion or all of said medical device that is expanded in said body passageway comprising:
providing said medical device; said medical device includes a balloon catheter; said balloon catheter includes a catheter body having a distal portion, a central portion, and proximal portion; said balloon catheter includes an inflatable balloon connected at or near said distal portion;
providing a diameter measurement device (DMD) is configured to measure a) said diameter of at least a portion or all of said medical device that is expanded in said body passageway, b) said cross-sectional area of at least a portion or all of said medical device that is expanded in said body passageway, and/or c) said volume of at least a portion or all of said medical device that is expanded in said body passageway; said DMD includes a plurality of excitation electrodes and a plurality of sensor electrodes; said plurality of excitation electrodes and said plurality of sensor electrodes are positioned inside said inflatable balloon; said plurality of excitation electrodes and said plurality of sensor electrodes are spaced from one another;
inserting said distal portion and a portion of said central portion of said catheter into said body passageway;
moving said distal portion of said catheter in said body passageway until said distal portion is positioned at a treatment area;
expanding said inflatable balloon at said treatment area; and
measuring a change in impedance by said DMD to determine at said treatment area a) said diameter of at least a portion or all of said medical device that is expanded in said body passageway, b) said cross-sectional area of at least a portion or all of said medical device that is expanded in said body passageway, and/or c) said volume of at least a portion or all of said medical device that is expanded in said body passageway, and wherein said change in impedance is at least partially related to said cross-sectional area and/or a pressure of said inflatable balloon.

2. The method as defined in claim 1, wherein said measuring a) said diameter of at least a portion or all of said medical device that is expanded in said body passageway, b) said cross-sectional area of at least a portion or all of said medical device that is expanded in said body passageway, and/or c) said volume of at least a portion or all of said medical device that is expanded in said body passageway is in real time or near real time.

3. The method as defined in claim 1, wherein said medical device includes a stent or prosthetic heart valve that is at least partially positioned about said inflatable balloon.

4. The method as defined in claim 1, wherein said distal portion of said catheter includes a temperature sensor.

5. The method as defined in claim 1, wherein said DMD includes multiplexed inputs.

6. The method as defined in claim 1, wherein said DMD includes a wireless transmitter that wirelessly transmits data to a remote location so that data can be stored and/or displayed at said remote location.

7. The method as defined in claim 1, wherein said DMD includes a reference container; said reference container has a) fixed and constant cross-sectional area along a longitudinal length of said reference container, b) a fixed and constant volume, c) a fixed and constant cross-sectional area along a longitudinal length of said reference container, and/or d) a fixed and constant cross-sectional shape along a longitudinal length of said reference container; said reference container includes a plurality of reference electrodes located in said reference container; a number, orientation, and/or spacing of said plurality of reference electrodes in said reference container is the same or substantially the same as a number, orientation, and/or spacing of said excitation and senor electrodes in said inflatable balloon.

8. The method as defined in claim 7, wherein said reference container has the same or similar a) longitudinal length, b) volume, c) cross-sectional area along a longitudinal length of said reference container, and/or d) cross-sectional shape along said longitudinal length of said reference container as said inflatable balloon when said inflatable balloon is partially or fully inflated.

9. The method as defined in claim 7, wherein said reference container is used to calculate a medium conductivity of a medium; said medium is used to inflate said inflatable balloon.

10. The method as defined in claim 9, wherein said medium includes a saline solution.

11. The method as defined in claim 1, wherein said step of measuring a change in said impedance includes impedance planimetry, wherein a constant voltage source having a high precision waveform is used across a known calibration resistor and impedance measurements are optionally sequenced across electrodes for segmental impedance measurements using the calibrated current value and voltages determined from each segment.

12. The method as defined in claim 1, further includes a flow sensor to provide information about a total volume of fluid into said inflatable balloon, and wherein said flow sensor can be optionally used to monitor a fluid flow rate and/or a total fluid volume delivered to said inflatable balloon to provide additional information for determining said diameter, said cross-sectional area and/or said volume of at least a portion or all of said medical device based on known relationships of said inflatable balloon.

13. The method as defined in claim 1, further includes a pressure sensor located distally in said inflation balloon and/or proximally in an inflating system, and wherein said pressure sensor is optionally used to facilitate determining said diameter, said cross-sectional area and/or said volume of at least a portion or all of said medical device by known pressure volume compliance calculations for said inflatable balloon and/or said inflating system.

14. A diameter measurement device system (DMDS) configured to measure a) a diameter of at least a portion or all of a medical device that is expanded in a body passageway, b) a cross-sectional area of at least a portion or all of said medical device that is expanded in said body passageway, and/or c) a volume of at least a portion or all of said medical device that is expanded in said body passageway; said DMDS comprises:
said medical device; said medical device includes a balloon catheter; said balloon catheter includes a catheter body having a distal portion, a central portion, and proximal portion; said balloon catheter includes said inflatable balloon that is connected at or near said distal portion;
a plurality of excitation electrodes and a plurality of sensor electrodes; said plurality of excitation electrodes and said plurality of sensor electrodes are positioned inside said inflatable balloon; said plurality of excitation electrodes and said plurality of sensor electrodes are spaced from one another;
a plurality of wires that provide current to said plurality of excitation electrodes and said plurality of sensor electrodes; each of said plurality of excitation electrodes and said plurality of sensor electrodes partially or fully encircle said plurality of wires; and
wherein said DMDS is configured to measure a change in an impedance derived cross-sectional area and pressure of said inflatable balloon as said inflatable balloon is inflated to determine a) said diameter of at least a portion or all of said medical device that is expanded in said body passageway, b) said cross-sectional area of at least said portion or all of the medical device that is expanded in said body passageway, and/or c) said volume of at least said portion or all of said medical device that is expanded in said body passageway.

15. The diameter measurement device system as defined in claim 14, wherein said medical device includes a stent or prosthetic heart valve that is at least partially positioned about said inflatable balloon.

16. The diameter measurement device system as defined in claim 14, wherein said distal portion of said catheter includes a temperature sensor.

17. The diameter measurement device system as defined in claim 14, wherein said DMDS includes multiplexed inputs.

18. The diameter measurement device system as defined in claim 14, wherein said DMDS includes a wireless transmitter that wirelessly transmits data to a remote location so that data can be stored and/or displayed at said remote location.

19. The diameter measurement device system as defined in claim 14, wherein said DMD includes a reference container; said reference container has a) fixed and constant cross-sectional area along a longitudinal length of said reference container, b) a fixed and constant volume, c) a fixed and constant cross-sectional area along a longitudinal length of said reference container, and/or d) a fixed and constant cross-sectional shape along a longitudinal length of said reference container; said reference container includes a plurality of reference electrodes located in said reference container; a number, orientation, and/or spacing of said plurality of reference electrodes in said reference container is the same or substantially the same as a number, orientation, and/or spacing of said excitation and senor electrodes in said inflatable balloon.

20. The diameter measurement device system as defined in claim 19, wherein said reference container has the same or similar a) longitudinal length, b) volume, c) cross-sectional area along a longitudinal length of said reference container, and/or d) cross-sectional shape along said longitudinal length of said reference container as said inflatable balloon when said inflatable balloon is partially or fully inflated.

21. The diameter measurement device system as defined in claim 19, wherein said reference container is used to calculate a medium conductivity of a medium; said medium is used to inflate said inflatable balloon.

22. The diameter measurement device system as defined in claim 21, wherein said medium includes a saline solution.

23. The diameter measurement device system as defined in claim 14, wherein a constant voltage source having a high precision waveform is used across a known calibration resistor and impedance measurements are optionally sequenced across electrodes for segmental impedance measurements using the calibrated current value and voltages determined from each segment.

24. The diameter measurement device system as defined in claim 14, further including a flow sensor to provide information about total volume of fluid in said inflatable balloon, and wherein said flow sensor can be optionally used to monitor a fluid flow rate and/or a total fluid volume delivered to said inflatable balloon provide additional information for determining said diameter of at least a portion or all of said medical device, said cross-sectional area of at least a portion or all of said medical device and/or said volume of at least a portion or all of said medical device based on known relationships of said inflatable balloon.

25. The diameter measurement device system as defined in claim 14, further including a pressure sensor located distally in said inflation balloon and/or proximally in an inflating system, and wherein said pressure sensor is optionally used to facilitate determining said diameter, said cross-sectional area, and/or said volume of at least a portion or all of said medical device by known pressure volume compliance calculations for said inflatable balloon and/or said inflating system.
